(19) United States
(12) Patent Application Publication
(10) Pub. No.: US 2025/0389706 A1
(43) Pub. Date: Dec. 25, 2025

(54) WATER ANALYSING DEVICE, MEASURING CELL AND METHOD FOR PHOTOMETRIC ANALYSIS

(71) Applicant: WATER MINILAB KFT., Andornaktálya (HU)

(72) Inventors: Péter SZOMBATHY, Budapest (HU); Bernadett CZIPÓ, Verpelét (HU); Ákos RACS, Budapest (HU); László DOMJÁN, Alsóörs (HU)

(73) Assignee: WATER MINILAB KFT., Andornaktálya (HU)

(21) Appl. No.: 18/707,452

(22) PCT Filed: Nov. 4, 2022
(86) PCT No.: PCT/HU2022/050079

§ 371 Date: May 3, 2024

(30) Foreign Application Priority Data
Nov. 4, 2021 (HU) P2100378

(51) Int. Cl. G01N 33/18; G01J 1/08; G01N 1/14; G01N 1/40; G01N 35/00; G01N 35/02; G01N 35/04; G01N 35/10

(52) U.S. Cl. CPC G01N 33/18; G01J 1/08; G01N 1/14; G01N 1/4077; G01N 35/0099; G01N 35/026; G01N 35/04; G01N 35/1002; G01N 35/1079; G01N 2001/4088; G01N 2035/00356; G01N 2035/00534; G01N 2035/0405; G01N 2035/042; G01N 2035/0475

(57) ABSTRACT

Water analysing device for the photometric analysis of samples taken from natural or artificial bodies of water. The water analysing device is suitable for onsite measurements without human intervention. Also, a water analysing system that includes the above water analysing device, a unit capable of GSM communication and a webserver. Additionally, a measuring cell for performing the photometric measurement of samples taken from natural or artificial bodies of water.

### THE FIELD OF THE INVENTION

The object of the present invention relates to a water analysing device and to the system associated with it, which serves for the measurement of several parameters, where at least some of the parameters may be measured photometrically. The object of the invention also relates to the use of the water analysing device.

### THE BACKGROUND OF THE INVENTION

Today, drinking water is obtained from two main sources. According to the one method it is nature itself that performs the purification of the water, in this case the water passes through numerous layers of gravel and sand, contamination gets stuck on these layers and the water becomes purified deep underground. According to the other method, the water obtained from a well is purified through a multistep filter. Only tap water of the appropriate quality is authorised for consumption, the quality of which may be checked using analytical tests.

Surface and subsurface waters, as well as rainwater are all classed as natural water. It is mainly from the latter that the surface waters, such as seas, lakes, rivers and streams, are formed. A proportion of surface waters and rainwater seeps through the layers of soil and goes to form groundwater above the impermeable layer. Water is called subsurface water after reaching the water table.

Wastewater is the water contaminated due to the effect of human activity, it can be of residential population, municipal waste water, agricultural or industrial origin. The quality of such waters is changed in terms of its physical, chemical and biological characteristics, and such waters contain water pollutant(s). These waters are especially infectious and environmentally polluting due to the organic materials and microorganisms in them. Wastewaters are generally returned to natural waters after purification. This is why it is exceptionally important for wastewaters to be properly treated and purified, thereby reducing the burden on the natural bodies of water. The water burden is understood to mean the emission of water pollutants into surface waters. The emission limit values relating to purified wastewater, i.e., the burden on the water represented by the pollutant, are regulated in authority decisions within the scope of ministerial decrees.

Usually, several different apparatuses and laboratory conditions are required in order to analyse multiple parameters of tap water, natural waters and wastewaters.

Today, the contamination of our waters is one of the leading environmental problems. The main sources of pollution are industry, agriculture, and the residential population. The protection of our natural waters demands continuous monitoring, which, among other methods, may take place using analytical measurements. The environmental and water authorities regularly take samples from our waters (living aquatic resources, natural bodies of water, artificial reservoirs, sewerage systems, drinking water, etc.), and in addition to their composition, the following parameters are usually measured: pH, chemical oxygen demand, biological oxygen demand, organic compound content, heavy metal content, etc. In parallel with this the authorities also measure municipal and industrial wastewater emissioners by taking samples from their drained wastewaters in order to determine the burden on the receiving water bodies (Csaba Demkó, The physical and chemical testing of natural waters, working paper, page 2, publisher: National Institute of Vocational and Adult Training, the identification number and target group of the content element: SzT-031-50, <https://www.nive.hu/Downloads/Szakkepzesi_dokumentumok/Bemeneti_kompetenciak_meresi_ertekelesi_eszkozrendszerenek_kialakitasa/14_1223_031_100915.pdf>).

An additional guide relating to the quality of Hungarian wastewaters is provided by Ministry of Environment and Water decree 28/2004. (XII. 25.) on certain rules pertaining to the limit values relating to the emission of water pollutants and their application. Part 1 of annex number of 1 of Ministry of Environment and Water decree 28/2004. (XII. 25.) contains the currently valid limit values relating to the quality of purified wastewater emitted by wastewater treatment plants according to Article 2, point 27 of Government decree 38/1995. (IV. 5.), by near-natural water treatment in the case of wastewater-producing settlements with fewer than 2000 population equivalents, by treatment performed by small water treatment plants, and by household water treatment plants. Basically, limit values are given for five components with respect to the quality of discharged water, which are the following: installed load capacity, dichromate chemical oxygen demand, biochemical oxygen demand, total suspended solids, total phosphorous, total nitrogen. Separate prescriptions concerning the given industry relate to industrial wastewaters.

Many water analysis devices or methods for analysing water are known of according to the state of the art.

Patent application number CN111487193A discloses a flow-through water analyser apparatus adapted for the quality analysis of several parameters, which, in addition to total phosphorous and nitrogen content, is also adapted for determining nitrogen content originating from ammonia and organic material content.

Utility model application number CN104634752A discloses an integrated, multi-parameter device for analysing the quality of mineral water via multiple parameters. The device consists of an all-in-one (hereinafter: ARM) type computer, a virtual keyboard and RS232 module, a wireless WIFI unit, a power supply transformer module, an ion potentiometer and a spectrophotometer. In the integrated, multi-parameter mineral water analyser device, the ARM computer is connected to the ion potentiometer and the spectrophotometer through the virtual keyboard and the RS232 module; the spectrophotometer is used for determining the absorbance of a water sample in the cuvette. The test data are entered into the ARM computer using the virtual keyboard and the RS232 module. In other words, the integrated, multi-parameter mineral water analyser device combines the spectrophotometer, the ion potentiometer and the ARM computer.

Patent application number U.S. Pat. No. 4,158,545A discloses a device for automatically analysing multiple chemical parameters. FIG. 2 illustrates the photometric detecting unit; every sample is subjected to an analysis consisting of four simultaneous parts.

Utility model number CN202442728U discloses a mobile device adapted for detecting that may be used to a broad extent, which contains a mobile detecting vehicle, a sampling device, a culturing box, a microscope, a water toxicity analysing instrument, a turbidity meter, a spectrophotometer, a water quality analysing instrument adapted for measuring multiple parameters, a purification basin, an air pollution measuring instrument, a noise detector instrument, a computer, an emergency detector reagent box, GPS (global positioning system). The aforementioned detecting apparatuses and devices are all located on the work platform in the mobile detecting vehicle. The device performs measurements in real time and in emergencies in construction projects, thereby ensuring the project complies with the legislation and prescriptions relating to environmental impact, preventing and mitigating ecological harm, and environmental pollution during the period of construction.

Patent application number US2020241028AA describes in detail an automatic analysing device. The device contains the following main units: an apparatus filling a reaction vessel, a sample unit, a sample dispensing device, a reagent unit, a reagent dispensing device, a reaction dish, a stirring machine, a measuring unit, a magnetic separation unit, a portable unit, and a control unit.

Patent application number IN201741016470A describes a device and system adapted for analysing water, more precisely for monitoring water pollution. In addition to being able to perform measurements of water quality, the invention is also able to determine the sources of the pollution. According to the section presenting the state of the art (page 3, lines 8 to 13), the apparatuses for monitoring water quality are most frequently the followings: spectrophotometers, BOD-analysers, turbidity meters, conductivity meters, COD-analysers, ion concentration meters, UV-VIS-spectrophotometers, UNICO-spectrophotometers, BOD-incubators, etc. In the application the list of disadvantages includes that it takes a long time to achieve a result, even as much as several days, and so the measured parameters may even change in the water over this time. In turn, this may lead to decisions in connection with water quality not being made in time. Parts of the invention include sensors that measure conductivity, ORP (oxidation-reduction potential) and dissolved oxygen content. The parts of the solution include a microcontroller, the task of which is to digitalise the signals, perform data transfer tasks, and manage the network, as well as a navigation unit, a wide-angle camera, and also a two-way communication unit, which contains a GSM modem and a 2.4 GHz wireless transceiver, where the communication unit transmits and receives the information for the shoreline-based computer unit.

The invention disclosed in patent application number CN107459190A describes an organic wastewater treatment system based on oxidation and a method examining its efficiency. In addition to many other components, the system contains a temperature sensor, a pH meter, conductivity meter, and a dissolved oxygen sensor. The data of the measured parameters are collected by the data collection card and forwarded to the computer, then the “LabVIEW” program running on the computer reads and analyses the data.

The object of utility model application number CN212432951U relates to a device adapted for analysing water quality and for measuring multiple parameters. The photometer of the optical pathway system contains a xenon lamp, a detector adapted for detecting the entire spectrum, a light detector, a colorimetric pool, and optical lenses. The device is used to determine phosphorous, total nitrogen, ammonia and nitrate content, chemical oxygen demand, and conductivity; it is also adapted for measuring several other parameters as long as these measurements do not interfere with each other.

Application number CN212255286U is also a utility model application that is able to monitor water quality in real time via an electrode module adapted for simultaneously measuring several parameters, and to determine spectrophotometer data, in addition it is also able to measure windspeed. The energy required for operation is provided by six solar cells. The measured parameters are the following: temperature, conductivity, dissolved oxygen, acidity and alkalinity, ammonia-nitrogen turbidity and chemical oxygen demand (the measurements listed do not require the addition of reagents). The invention is used for efficiently monitoring the change in water quality in real time, and is capable of raising the alarm in the case of emergencies.

Patent application number DE102016109472A1 describes a method for the simultaneous determination of the amounts of several components of mixtures. The steps of the method are as follows: a) the determination of the optical molecular spectrum of the components to be analysed from a multi-component mixture and/or suitable pure material in order to obtain a spectral “imprint” for the pure material or mixture; b) the creation of the calibration model for every component of the sample to be tested in order to be able to test various concentrations and interactions; c) the performance of multivariate data analysis to create the calibration model for the individual materials.

Utility model application number RU172097U1 discloses a photometric device adapted for the analysis of pollutant crude oil derivatives in water using broad spectrum UV radiation.

Patent application number WO9920789A1 describes a sensitive detecting method that is adapted for the determination and measurement of one or more analytes. The method of identifying the analyte or analytes is that they are linked to a light-dispersing particle. The method and the device were designed in order to maximise the detecting of the light that is dispersed only by these particles, meaning that there is no need for a microscope or other imaging system. The light source used may be monochromatic or polychromatic, coherent or non-coherent; it does not have to be polarised, and may be low intensity, such as a LED or 12-watt incandescent bulb.

The photometric signal is influenced by the variation in the intensity of the light source and by the fluctuation in the sensitivity of the photodetector. If the aforementioned causes are not reduced or eliminated, the data may not be treated as being reliable. Solving this problem is very expensive and a good result is not guaranteed either. On the other hand, the absorbance and concentration of the measured component are directly proportionate to each other, nevertheless the measurement may be burdened with measurement and experimental errors, which they seek to overcome by recording a calibration curve. However, the calibration curve only relates to the given device, in other words it cannot be universally applied, in addition it is necessary to periodically check and possibly correct it. An additional analytical problem may come from the shifting of the baseline, and in order to correct this the well-known two-wave method and the baseline method are used, well known by the person skilled in the art. Although the aforementioned methods may be said to be good, it is assumed that the ratio of the intensity of the light source does not change at a reference wavelength and at a measurement wavelength. The profile of the spectrum of the light source is stable within a short period of time, but over a longer amount of time this intensity ratio changes, and so baseline correction is required. If the light source is replaced, then the baseline has to be corrected once again. The objective of the invention disclosed in patent application number U.S. Pat. No. 5,387,971A is to provide a method for measuring concentration that is precise, reliable, reproducible, does not require baseline correction and requires minimal cell cleaning. In order to overcome the problems disclosed above complementary modulated cells are used (sample and reference cells). The concentration in the sample may be determined on the basis of the differences of the measured absorbance.

Patent application number WO8706008A2 deals with an automated, multipurpose analytical chemical processing centre and with a laboratory workstation. The workstation is adapted for performing programmable spectrophotometric measurements; it processes the results (creating an interactive connection with a remote computer) and records the data.

Patent application number JP2021047140A discloses an automatic analyser that performs the quality and quantity analysis of biological samples (such as blood and urine). The parts of the analyser are as follows: reaction vessel(s) (1 to 8 vessels), spectrophotometer, thermostatic chamber with circulation flow pathway, light source, concentration sensor, and a computer suitable for controlling.

Patent application number CN103785314A discloses a mixer and analyser using a photometric principle adapted for examining specific components of solutions (such as water) or studying chemical reaction dynamics. This application contains a unit adapted for stirring, but makes no mention of the use of multiple cuvettes.

Utility model number CN213517175U deals with a device adapted for the preparation of samples. A cuvette is located on the sample plate, and the sample dispensing device is guided to this. The apparatus also includes a unit adapted for cleaning the cuvette.

Patent application number U.S. Pat. No. 4,431,924A deals with a multi-channel analysing device provided with a code system.

The water analysing apparatuses for measuring multiple parameters according to the state of the art are usually adapted for simultaneously measuring multiple parameters by having more than one analysing instrument or detector installed in them.

The objective of the present invention is to provide a water analysing device that is adapted for photometrically measuring multiple parameters and that is free of the disadvantages of the solutions according to the state of the art. The water analysing device according to the present invention has multiple cuvettes and is adapted for performing precise, onsite photometric measurements without human intervention, thereby it is more economic than the water analysing devices currently known of.

It was recognised that if the known spectrophotometers are transformed in such a way that the device contains more than one cuvette, and the cuvettes containing the samples to be measured using the spectrophotometer are placed in the measuring cell using a robotic arm, then we obtain a device operating on a photometric principle adapted for performing automated photometric measurements without human intervention.

### A BRIEF DESCRIPTION OF THE FIGURES

FIG. 1 depicts the schematic structure of the water analysing device according to the invention.

FIG. 2 depicts the structure of the bore for accommodating the cuvettes.

FIG. 3 shows the structure of the measuring cell.

### BRIEF DESCRIPTION OF THE INVENTION

The object of the present invention relates to a water analysing device for the photometric analysis of samples taken from natural or artificial bodies of water, and which water analysing device contains the followings:
a frame, covered by thermal insulation, in which fans are used to ventilate the interior space;
tubes between the individual elements of the water analysing device for transporting the fluids;
sampler for taking samples from the natural or artificial bodies of water;
one or more sampling pumps for transporting fluids, for sucking up the water from the sampler;
one or more filters connected to the sampler and the sampling pump for filtering out the shaped bodies to be found in the samples;
one or more buffer tanks connected to the sampling pump;
one or more needles or dispensing units for dispensing the filtered sample, the reagents and the solvents;
one or more sample dispensing pumps connected to the buffer tank;
one or more reagent dispensing pumps connected to the reagent tanks;
one or more sample and reagent dispensing positions connected to the sample and reagent dispensing pumps;
one or more cuvettes;
robotic arm for moving the one or more cuvettes, on which robotic arm there is a gripping arm, and which gripping arm is adapted to grasp the cuvettes;
replaceable tray for storing the one or more cuvettes;
a stirring unit for stirring the sample and the reagents;
a measuring cell for the photometric analysis of the sample in the one or more cuvettes;
source of electricity for operating the components operating with electricity.

The present invention also relates to a water analysing device in which there is more than one cuvette.

According to a preferable embodiment of the present invention the sampler preferably has an adjustable length and/or a silicone tube connects it to the one or more filters via a sampler pump.

According to another preferable embodiment of the present invention the filter insert of at least one of the one or more filters is metal.

According to an additional preferable embodiment of the present invention the covering is provided with thermal insulation.

According to a preferred embodiment of the water analysing device according to the present invention the number of cuvettes is between 120 and 240.

According to an additional preferred embodiment at least some of the cuvettes in the water analysing device are sealed with a septum or a cap.

According to another preferred embodiment the gripping arm of the water analysing device is provided with a light source and a sensor located opposite each other in such a way that when the cuvette is grasped the light emitted by the light source gets to the sensor via the cuvette.

The present invention also relates to a water analysing device that contains a heated digester.

The present invention also relates to a water analysing device that contains a cap screwing unit adapted for removing and replacing the cap of the cuvette.

The present invention also relates to a water analysing device that has a filter and the filter insert of the filter has holes with a diameter of 25-80 μm. The present invention also relates to a water analysing device that has two filters, where the filter insert of the first filter according to the flow of liquid has holes with a diameter of 25-80 μm and the filter insert of the second filter has holes with a diameter of 0.4-25 μm. The one or more filters are provided with a pressure sensor.

According to another preferred embodiment the replaceable tray of the water analysing device contains three plates, where the lower plate is continuous in structure, and the two upper plates contain bores for accommodating the cuvettes, and said bores have a conical shape.

According to a preferred embodiment of the water analysing device according to the present invention the number of needles, or of the dispensing positions is between 1 and 10, preferably 6.

According to a preferred embodiment of the water analysing device according to the present invention the number of pumps is between 2 and 10, preferably 6.

According to a preferred embodiment of the water analysing device according to the present invention the number of wavelengths measured in the measuring cell is between 1 and 8, preferably 4.

A measuring cell is located in the water analysing device according to the present invention, which is for performing the photometric measurement of samples taken from natural or artificial bodies of water, and which contains the followings:
a broad spectrum light source;
a light source aperture positioned directly in front of the light source;
an aspherical collimator lens positioned in front of the light source aperture;
an optical filter changer;
a semi-transparent mirror;
a measuring branch aperture located behind the semi-transparent mirror;
a cuvette located behind the measuring branch aperture;
a transmission detector of a size larger than a measuring beam located behind the cuvette;
a reference aperture positioned at 45° next to the semi-transparent mirror;
a reference detector of a size greater than a reference beam behind the reference aperture.

Furthermore, the present invention also relates to a method for performing the photometric analysis of samples taken from natural or artificial bodies of water, where the analysis is performed with the water analysing device according to the above, and which method contains the following steps:
a) before sampling, removing the stagnant water from the water analysing device, then rinsing the sampler with adjustable length and the one or more filters with the sample to be measured;
b) sampling;
c) filtering the sample obtained in step b) through the one or more filters;
d) the rinsing of the buffer tank supplied with a level indicator, the sample dispenser and the section between the two with the filtered sample to be measured obtained in step c);
e) grasping the cuvette with the gripping arm and checking if the cuvette is open or sealed;
f) the transporting of the cuvette gripped by the grasping arm with the robotic arm to the given position, if necessary, the screwing off of the cap of the cuvette sealed with a cap and the transporting of the cuvette to the sample dispensing position or pushing the cuvette sealed with a septum onto the sample dispensing needle;
g) optionally the dispensing of demineralised water or other reagent into the cuvette at the sample or reagent dispensing position;
h) screwing the cap onto open cuvettes without caps;
i) homogenising the sample in the cuvette;
j) optionally digesting the sample in the cuvette at a temperature higher than the ambient temperature using the heated digester;
k) after a predetermined waiting time has elapsed the cuvette is placed in the measuring cell by the gripping arm and the robotic arm, where the photometric measurement of the sample in the cuvette takes place.

According to an even more preferable method of implementation of the invention the temperature applied in step i) is between 120° C. and 180° C., preferably 150° C., and/or the digesting duration is 30-140 minutes, preferably 30 minutes.

In the course of the method according to the present invention a clean cuvette is preferably used for each sample.

According to another preferred method of implementation using the method according to the present invention the parameters selected from the following list are determined: phosphate ions; nitrite ions; nitrate ions; ammonium ions; chemical oxygen demand, dissolved hydrogen sulphide, sulphides.

The present invention also relates to the water analysing system that includes the above water analysing device, a unit capable of GSM communication and a webserver.

### DETAILED DESCRIPTION OF THE INVENTION

Within the scope of the present specification if a numerical value is given it is understood that the last digit of the given number shows the precision of the given value in accordance with the rules of rounding. In other words, for example, 120 is understood to mean the range of 115 to 124.

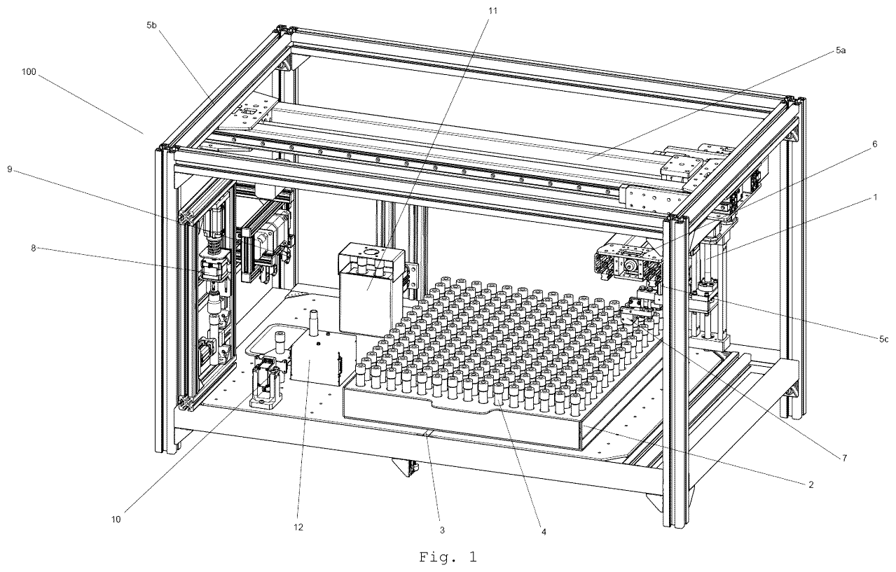

FIG. 1 shows a possible schematic structure of the water analysing device 100 according to the present invention. In the interest of easier comprehension, the frame and the thermal insulation covering it (for the sake of simplicity these two are referred to together as the housing), the fans, the tubes and the sampler located in the thermal insulation, have not been depicted.

The water sampling device 100 contains a filter 1, a buffer tank (not specifically illustrated in FIG. 1), additionally six pumps (these are not specifically illustrated in FIG. 1), which are for transporting the liquids within the water analysing device 100. It is necessary to move the following liquids in the water analysing device 100: sample taken from artificial or natural bodies of water, the solvent required to dilute the sample into the measurement range (such as water), and the reagents required for performing the measurement. Different types of pumps are used for moving the various liquids, which are the followings: sampling pumps; sample dispensing pumps; reagent dispensing pumps. The water analysing device 100 also includes a replaceable tray 2 for accommodating the cuvettes 4, in the case of the replacement of which replaceable tray 2 it may be moved out from the housing via the first rail 3. The cuvettes 4 may be found in the replaceable tray 2. The robotic arm 6 travels above the replaceable tray 2 on the second rails 5a, 5b, 5c, which robotic arm 6 is provided with a gripping arm 7, and it is the task of the robotic arm 6 to grasp one of the cuvettes 4 using the gripping arm 7, or to transport the cuvettes 4 for preparing the samples and/or for digestion, and also to transport them to the photometric measurement location. The cuvettes 4 sealed with a cap or a septum may be differentiated from the open cuvettes 4 with the use of the light source and sensor (not specifically indicated in FIG. 1) built into the grasping arm 7. A light source and sensor are located opposite each other in the gripping arm 7 of the water analysing device 100, due to this when the cuvette 4 is grasped the light emitted by the light source passes into the sensor through the cuvette 4. If a cuvette 4 is sealed with a cap, then the light is unable to pass from the light source into the sensor, therefore the robotic arm 6 of the water analysing device 100 transports the sealed cuvettes 4 into the cap screwing unit 8, which removes the cap of the cuvette 4. The light source and sensor built into the gripping arm 7 are used to check whether the screwing on and off of the cap was successful. The robotic arm 6 takes the cuvettes 4 without caps or sealed with a septum to the sample and/or reagent dispensing position without the involvement of the cap screwing unit 8. In addition, six needles or dispensing units (not specifically illustrated in FIG. 1) may be found in the water analysing device 100, which are used for dispensing the sample, other reagents or solvents at the sample and reagent dispensing position 9. The solvents and/or the reagents are stored in separate reagent and solvent tanks used especially for this purpose (these are not shown in FIG. 1), the dispensing of the liquids stored in them is possible using the pumps adapted for dispensing the reagents. The water analysing device 100 also includes a stirring unit 10 and one measuring cell 11. The water analysing device 100 also includes a heated digester 12. The replaceable tray 2 contains conically shaped bores 13.

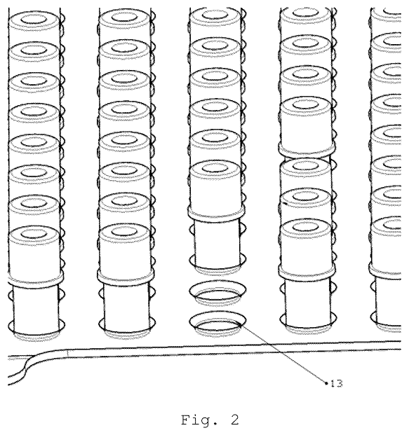

The water analysing device 100 is positioned in an environmentally safe position next to the body of water to be tested (wastewater or natural water), if possible, on a flat, solid base, in such a way that the water analysing device 100 is able to take a sample from the body of water to be analysed through a silicone tube secured to a telescopic sampler using one of the sampling pumps. Before sampling, the stagnant water that has got into the silicone tube secured to the telescopic sampler during the waiting time between the individual measurements is removed. The silicone tube secured to the sampler is insulated, and may be heated in order to prevent freezing. The sample is sucked through the silicone tube secured to the sampler using one of the sampling pumps and the filter 1 is rinsed using the sample to be measured. A buffer tank is connected to the sampling pump (not depicted in FIG. 1), which is open to the air, provided with an overflow and provided with a water level sensor. The function of the buffer tank is to provide identical amounts of sample, which the sample dispensing pump transports. The reason for this is that in the case of the creation of pressure or a vacuum, the pump would not transport the precisely set amount. After the telescopic sampler and the filter 1 have been rinsed with sample, the sample to be measured is sucked into the filter 1 and filtered in order to filter out the shaped bodies. The filter 1 is cylindrical in shape, as a result of which the filter insert may be easily replaced. The pressure may be measured on the shell side of the filter 1, so the filter 1 may be checked for blockages or the extent of the blockage, and its cleaning or replacement may be scheduled. A ball valve may be found on the base of the filter 1, which if opened and the valve at the sampler is closed, then the filter 1 may be washed off into the body of water, i.e., into the sampling location. Compressed air is used for this, which is fed in via the solenoid valve located on the top of the filter 1. The compressed air is supplied to the water analysing device 100 by a compressor (not separately shown in FIG. 1). The compressed air forces out the sample located in the filter 1 at great speed, and so blows off the contamination from the filter insert of the filter 1, after the water content of the sample has been removed the compressed air loosens the solid contamination settled on the surface of the filter insert. The filter insert located in the filter 1 has holes with a diameter of 25-80 μm, which may be used for pre-filtration, the diameter of the filter insert is suitable for filtering the shaped bodies in the larger size range to be filtered out of the sample. (It should be noted that an embodiment is conceivable that contains two filters 1. In this case the filter insert of the second filter 1 has holes with a diameter of 0.4-25 μm, which is suitable for filtering out the elements in the smaller size range passing through the first filter 1.) An extraction connection may be found in the upper part of the internal space of the filter (not indicated in FIG. 1), through which the filtered sample may be pumped into the buffer tank, which is first rinsed with filtered sample, and then filled up with it. Two level indicators measure the level of the filtered sample in the buffer tank, the sampling pump operates until the level of the sample reaches the level of the two-level indicators. As a result of the air pipe end located on the buffer tank the buffer tank is at atmospheric pressure, which ensures that the filtered sample is at atmospheric pressure, in other words so that the pump pumps identical amounts of sample even if there is a change in water level, and the air pipe end also acts as an overflow. The amount of sample that is in excess of the overflow passes out of the water analysing device 100. The pressure sensor located between the external shell of the filter 1 and the filter insert measures the pressure drop in the shell side of the filter 1. The drop in pressure may allow conclusions to be drawn about the condition of the filter 1, about the extent to which it is blocked, thereby its cleaning or replacement may be scheduled. Optionally, the back-washing of the filter 1 may be performed with compressed air, for this a compressed air connector is used. Filtered sample is pumped out of the buffer tank via its extraction connection to the sample and reagent dispensing position 9, more precisely into the secured sample dispensing needle or sample dispenser, by one of the sampling pumps (e.g., a stepper motor peristaltic pump). Before measurement is performed, the section between the sample and reagent dispensing position 9 and filter 1 is rinsed with filtered sample in order to avoid cross-contamination. Before dispensing the sample, the light source and sensor built into the gripping arm 7 are used to determine whether the cuvette 4 is open or sealed. The basis of this test is that the light beam emitted by the light source does not pass through the cap into the sensor positioned opposite to it. If only sealed cuvettes 4 have been placed in the water analysing device 100, and on the basis of the test a cuvette 4 still appears to be open, then the robotic arm 6 provided with the gripping arm 7 replaces the faulty cuvette 4 back into the replaceable tray 2 and picks out a new cuvette 4. If on the basis of the test a cuvette 4 is sealed with a cap, then the robotic arm 6 provided with the gripping arm 7 transports it to the cap screwing unit 8, which removes the cap of the cuvette 4, then following this the robotic arm 6 transports the cuvette 4 without its cap to the sample dispensing needle or sample dispenser. In order to screw off the cap the base of the cuvette 4 must be held firmly in one position, for this gripping clamps have been built into the cap screwing unit 8 that a pneumatic slave cylinder clamps onto the cuvette 4. A metal clamping component operated by a stepper motor secured to a bogie is used for screwing the cap of the cuvette 4 off and on. When screwing off the cap, the cap remains in the metal clamping component. A slave cylinder is installed for pushing down and pulling up the stepper motor, with this the metal component gripping the cap is removed in the case of screwing off or screwing on, or pushed onto the cuvette 4. In order to overcome the effect of the rise of the thread caused by the screwing a spring was built in between the slave cylinder and the bogie, thereby the rigidity of the system is partially eliminated and is able to tolerate movements in this direction, furthermore the forcing of the cap onto the cuvette 4 depends on the strength of the spring. The use of the spring is preferred because in order to precisely perform movements requiring such screwing on and off it would be necessary to precisely set the air pressure in the unit performing this, which would unnecessarily increase the production and servicing costs of the water analysing device 100. Using the light source and sensor built into the gripping arm 7 it is also possible to check whether the screwing on and off the cap was successful. If the cuvette 4 is sealed with a septum, then the gripping arm 7 grasps the cuvette 4 filled in advance with reagents in such a way that it leaves its top free for the sample dispensing needle. There are 240 pieces of sealed (with a septum or cap) or open cuvettes 4 arranged in the replaceable tray 2 in the water analysing device 100. The base of the replaceable tray 2 constitutes a continuous surface on which the cuvettes 4 rest, its two upper plates contain bores 13 for accommodating the cuvettes 4. The edges of the bores 13 for accommodating the cuvettes 4 in the replaceable tray 2 are conically shaped becoming narrower from the direction of arrival of the cuvettes 4 (see FIG. 2), which helps the robotic arm 6 position the cuvettes 4 in the given bores 13 of the replaceable tray 2. The replaceable tray 2 is positioned in the water analysing device 100 in such a way that it may be removed through the door located on the side of the water analysing device 100 (not depicted) via the first rail 3, thereby making it easier to replace the pre-filled cuvettes 4 without removing the thermally insulated housing. The frame provided with thermal insulation, which for the sake of simplicity is called a housing, is waterproof, the air may be sucked out with the fans on it, and so in this way fresh air enters the housing from below, thereby ensuring that the air in the internal space does not overheat. A filter is placed on the fans, with the one fan air may be forced inside the housing, and with the other fan air may be sucked out of there, and as a result of the filters the internal space of the water analysing device 100 remains clean. Sealed cuvettes 4 are preferably used in the water analysing device 100, and the method of sealing may be with a septum or a cap. The septum hermetically seals the cuvette; however, the sample dispensing needle is able to pierce through the septum, in other words, in this case there is no need to use the cap screwing unit 8. The sample is transported into the open cuvette 4 (after the removal of the cap/already open) or into a cuvette 4 sealed with a septum using one of the sample dispensing pumps. If sealed cuvettes 4 are used, this stops any reagent or reagents filled into them in advance from evaporating, their composition changing, and protects the reagents from adverse environmental impacts (such as moisture) and also prevents the reagents from being spilled out. A separate, clean cuvette 4 is used for each photometric measurement, as in the case of the repeated use of the same cuvette surfaces there is the risk of a biofilm, alga or chemical deposits building upon them. It should be noted that clean cuvettes 4 are understood to mean cuvettes 4 that have been washed at least twice with tap water and then at least twice with demineralised water. When replacing the replaceable tray 2 the cuvettes 4 may be collected, centrally cleaned and used after being refilled. The cuvettes 4 are removed from the replaceable tray 2 by the gripping arm 7. The robotic arm 6 operating the gripping arm 7 moves similarly along the “x” and “y” axes as implemented in 3D printers in the way known to the person skilled in the art. The robotic arm 6 transports the cuvette 4 on the second rails 75a and 5b to the sample and reagent dispensing position 9. The head part of the robotic arm 6 moves on the “z” (vertical) axis (second rail 5c), and positions the cuvette 4 under the sample dispensing needle or, in the case of cuvettes 4 sealed with a cap, it positions the cuvette 4 now without its cap under the sample dispensing silicone tube and positions it for sample dispensing, then in the case of the movement of cuvettes 4 sealed with a septum it pushes it onto the sample dispensing needle. As stated further above, the sample is transported by one of the peristaltic sample dispensing pumps through the sample dispensing needle or sample dispenser into the cuvette 4. In the case of dispensing a smaller amount of sample through a septum it is sufficient to use the sample dispensing needle, however when dispensing a larger amount of sample through a septum it becomes necessary to use another, so-called venting needle due to the pressure formed in the sealed cuvette 4. Apart from the two previous types of needles, it is also possible to dispense the following materials: demineralised water, which is required for diluting concentrated samples; in addition, the dispensing of additional reagents may also be performed using needles or reagent dispensers, and these are stored in the reagent storage tanks. Each needle or silicone tube has a separate pump. The amount of dispensed sample varies depending on the measured parameter. If required for the specific measurement, additional reagents may also be dispensed (such as phosphate-phosphorous or ammonia-nitrogen reagents) or the sample may be diluted (in the case of an overly concentrated sample, the sample may be diluted to the measurement range with the addition of demineralised water), one of the reagents dispensing pumps may be used for adding the reagents. If the cuvette 4 was sealed with a cap, then the cap is screwed on with the gripping arm 7 and the robotic arm 6 with the help of the cap screwing unit 8. The stirring unit 10 is for homogenising the sample, the reagent and/or solvent in the cuvette 4, which may be a magnetic stirrer (in this case a magnetic stirring bar is placed in the cuvette 4 containing the sample), a shaker, or the sample may be homogenised by rotating it in two directions while upside-down, which is performed by a servo motor rotating a rotation arm. It should be noted that the magnetic stirrer, the shaker or upside-down rotation may be used interchangeably. The duration of the homogenisation (stirring) and the subsequent waiting time varies depending on the measurement performed (see Table 1 below). In the case of homogenisation, by using upside-down rotation, the bubbles on the wall of the cuvette 4 disturbing the spectrophotometric measurement may be eliminated (the bubbles diffract, scatter light and so make the measurement of light absorption imprecise). A heated digester 12 has also been installed due to the samples that require digestion, which may be heated with a heating insert or heating wire, and is made from aluminium; its temperature may be monitored using a thermometer, and it may be moved under an extractor hood on a rail or by using a pneumatic solution (not specifically indicated in FIG. 1) and it is surrounded by a housing as thermal insulation (not specifically indicated in FIG. 1). The purpose of the housing is to increase the efficiency of the fan extracting the waste heat (not specifically indicated in FIG. 1). The housing has a thermal insulation layer, due to the extraction fans built in, the surplus warm air formed during digestion may be removed. The digestion of the samples is in all cases performed after the samples are homogenised. Because of the digestion the sample preparation and photometric measurement processes are intermittent in the laboratories according to the present practice, however, both steps may be performed in a single location in the water analysing device 100 according to the present invention. For example, digestion is required by the photometric measurements relating to organic materials (e.g., COD, i.e., measurements relating to chemical oxygen demand). According to standards and manufacturer recommendations such digestion is performed at approximately between 120-180° C., preferably at a temperature between 149-155° C., and for a duration of 30-140 minutes. Experience shows that, contrary to the prescriptions in the standard and manufacturer recommendations, durations even shorter than 120 minutes are sufficient for the digestion of wastewaters with a low organic material content. It is preferable that for the digestion the cuvette 4 is sealed with an unused septum or cap. It is preferred if a septum used for digestion is not used again for further digestion, but otherwise used a maximum of five times for measurements not involving digestion. The longer use of the heated digester 12 would significantly increase the internal temperature of the water analysing device 100, therefore an extractor hood with a fan has been installed in the interest of ensuring the appropriate internal temperature. In warm weather the waste heat produced by the heated digester 12 (e.g., in summer in Europe) is extracted, and in cold weather (e.g., in winter in Europe) the water analysing device 10 can be tempered with this heat. In other words, the temperature of the water analysing device 100 is controlled and can be maintained between 20-30° C., which is suitable for the photometric measurements that may be performed in the water analysing device 100. Even heat distribution within the water analysing device 100 is achieved by using a circulation fan (not specifically indicated in FIG. 1). After the given waiting time has elapsed, the prepared sample is measured photometrically in the measuring cell 11. The data measured by the measuring cell 11 is sent to the webserver, where further calculations are performed, and then the final results are displayed in both table and graphic format. On completion of the measurement the filter 1 is backwashed and any water in the water analysing device 100 is removed if no more measurements are to be performed with the given sample. The spectrophotometer is an optical material testing device with which the change in colour of the substantially monochromatic light in the water due to the effect of the presence of the material to be detected can be measured. The common aspect of the many technical solutions used in these devices is that the atomic-molecular level energy transitions of the materials are quantised.

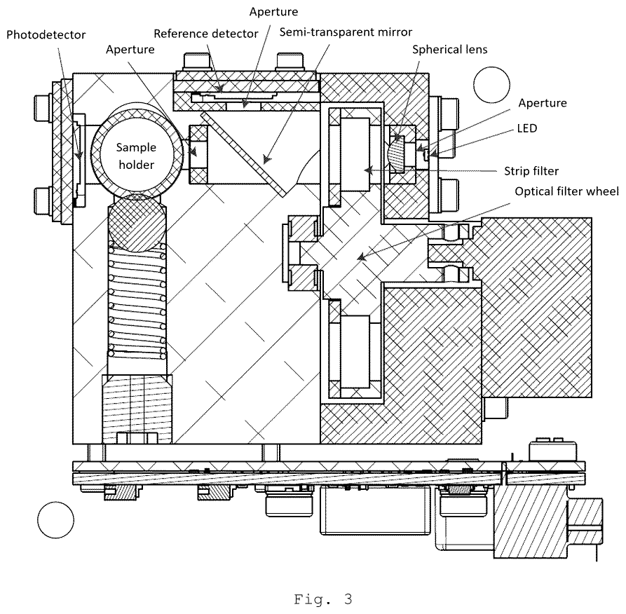

A measuring cell 11 (see FIG. 3 for its structure) is to be found in the water analysing device 100 according to the present invention, which is for performing the photometric measurement of samples taken from natural or artificial bodies of water, and which contains the followings: a broad spectrum light source; a light source aperture positioned directly in front of the light source; an aspherical collimator lens positioned in front of the light source aperture, an optical filter changer is located to the side of this; a semi-transparent mirror positioned behind a band pass; a measuring branch aperture located behind the semi-transparent mirror; a cuvette 4 located behind the measuring branch aperture; a transmission detector of a size larger than a measuring beam located behind the cuvette; a reference aperture positioned at 45° next to the semi-transparent mirror; a reference detector of a size greater than a reference beam behind the reference aperture.

An LED light source (not indicated in FIG. 1) of optional wavelength is used in the measuring cell 11 for performing the photometric measurements in the water analysing device 100, and the photometric measurement is performed with a light intensity detecting device (not depicted), which is preferably a phototransistor or a photodiode. The intensity of the light passing through the sample is detected using the light intensity detecting device, and from this the amount of light absorption may be concluded, which is proportional to the concentration of the material to be measured in the sample. The light source is preferably an LED light source, which means Light Emitting Diode. Usually, the spectrophotometers of water analysing devices contain an LED light source that emits light that is nearly monochromatic. Contrary to this, the broad spectrum light source used in the water analysing device 100 is an LED emitting optional wavelength light, from which the nearly monochromatic light beam is formed by using optical filters. The function of the semi-transparent mirror in the measuring cell 11 is to split the light beam in the 90%-10% ratio, to guide the 10% into the reference detector and the remaining 90% through the sample and into the transmission detector. If the light source of the measuring cell 11 is switched off and on several times one after the other, several photometric measurements may be performed, the values of which are averaged. The background noise appearing during the photometric measurement may be subtracted by measuring the dark current.

The power supply required for performing the photometric measurement in the water analysing device 100 may be provided from the mains network or with a solar cell (not illustrated). The solar cell is a good choice because it is a power supply independent of the mains, as there is not always a mains supply next to natural bodies of water. It should be noted that for the preparation of samples requiring digestion, and for the heating of the water analysing device 100 in the winter months in certain cases a lower capacity solar cell would not necessarily ensure a sufficient power supply, in such cases another power supply (e.g., mains electricity) must be provided, furthermore the water analysing device 100 may also obtain the power for performing the measurements from other power sources apart from the solar cell (such as the mains supply, which in Europe is 230 V).

The water analysing system (not depicted) according to the present invention includes the water analysing device 100, control electronics controlled by a microcontroller, in which there is a unit capable of performing GSM communication, for which an external antenna may be attached, and a webserver. The data measured by the water analysing device 100 are sent to an external webserver with a global mobile telecommunication device, i.e., GSM, where the data are processed, and the data are stored using e.g., a cloud-based service. After logging in the user may access the data with a mobile device, personal computer (PC), and even using an internet browser. It is due to the GSM connection that the water analysing device 100 itself informs the webserver that it will soon be necessary to replace the replaceable tray 2 and/or the insert of the filter 1, which the users obtain ready packaged. Calculations may be performed on the data stored on the webserver, and the results may be displayed in table or diagram format. On the basis of the measured data it is possible to make predictions, and, if necessary, send warnings and intervention proposals as well. When processing the data, the current weather conditions are taken into consideration, while when making predictions the expected weather is taken into consideration, the reason for this is that the weather conditions have an impact on water quality and on the chemical parameters measured. Due to being able to forward the analysed data and the results and send warning notifications, a high degree of decision support is provided, which makes the work of the operators of wastewater treatment plants much easier.

The measurement principle used in the water analysing device 100 is that during the photometric measurement the concentration of the examined component in the sample may be determined from the amount of light absorbed in the sample on the basis of the factory, pre-measured calibration line. Standard solutions are used in order to record the calibration line, in addition the standard solutions also serve for correcting the measured values, then these may be used to check the precision of the water analysing device 100, and maintenance may be scheduled in the case of any deviation.

If there is no possibility to send the data with a GSM communication unit to the webserver, the data may be saved onto a data carrier, e.g., an SD card.

On completion of the photometric measurement the robotic arm 6 is used to replace the cuvette 4 onto the replaceable tray 2. Following this the water is removed from the water analysing device 100, which includes the section between filter 1 and the sample and reagent dispensing position 9, and the section before the filter 1, as well.

The photometric measurements that may be performed with the water analysing device 100 and the data connected to the implementation of the measurement are contained in table 1.

| | | | | | Amount of | |
|---|---|---|---|---|---|---|
| | | | | | Sample | |
| | | | | | required | |
| | | | | | for | |
| Measured | Measurement | | Wavelength | | measurement | Sample |
| parameter | range | | (nm) | Reagent | (ml) | preparation |
| Phosphate | 0-0.83 | ppm* | 525 | Hanna | 10 | 2 minutes |
| ion (PO₄³—P) | | | | Instruments, | | stirring + 3 |
| | | | | HI 93713-01 | | minutes |
| | | | | reagent | | waiting |
| | | | | powder | | |
| Phosphate | 0.23-10 | ppm | 530 | Hach, 2 | 10 | 8 droplets of |
| ion (PO₄³—P) | | | | solutions | | reagents A |
| | | | | | | and B added + |
| | | | | | | 10 minutes |
| | | | | | | waiting |
| Nitrite- | 0-600 | ppb | 470 | Hanna | 10 | 15 sec |
| nitrogen | | | | Instruments, | | stirring + 15 |
| (NO₂—N) | | | | HI 93707-01 | | minutes |
| | | | | reagent | | waiting |
| | | | | powder | | |
| Nitrate- | 0-30 | ppm | 555 | Hanna | 10 | 10 sec |
| nitrogen | | | | Instruments, | | intense, 50 |
| (NO₃—N) | | | | HI 93728-01 | | sec careful |
| | | | | reagent | | stirring + |
| | | | | powder | | 4.5 minutes |
| | | | | | | waiting |
| Ammonium- | 0-10 | ppm | 470 | Hanna, 2 | 10 | 4 droplets of |
| nitrogen | | | | solutions, | | reagents A |
| (NH₄—N) | | | | HI700-25 | | and B added |
| | | | | | | then stirring + |
| | | | | | | 3.5 minutes |
| | | | | | | waiting |
| Ammonium- | 0.2-2.5 | ppm | 425 | Hach, | 10 | 1 minute |
| nitrogen | | | | solution | | waiting |
| (NH₄—N) | | | | | | |
| COD LR** | 0-150 | ppm | 430 | Lovibond, | 2 | 120 minutes |
| | | | | solution, | | digestion |
| | | | | 2420720 | | (150° C.) + |
| | | | | | | stirring, ~20 |
| | | | | | | minutes |
| | | | | | | cooling |
| COD MR** | 0-1500 | ppm | 610 | Lovibond, | 2 | 120 minutes |
| | | | | solution, | | digestion |
| | | | | 2420721 | | (150° C.) + |
| | | | | | | stirring, ~20 |
| | | | | | | minutes |
| | | | | | | cooling |
| COD HR** | 400-4000 | ppm | 610 | Macherey- | 2 | 120 minutes |
| | | | | Nagel, | | digestion |
| | | | | solution, | | (150° C.) + |
| | | | | REF 985011 | | stirring, ~20 |
| | | | | | | minutes |
| | | | | | | cooling |
| COD MR | 0-1500 | ppm | 600 | Obuda | 3 | 30 minutes |
| | | | | University | | digestion |
| | | | | | | (150° C.) + |
| | | | | | | stirring, ~20 |
| | | | | | | minutes |
| | | | | | | cooling |

the measurement parameters photometrically measurable by the water analysing device 100 and the related sample preparation;
*ppm = mg/l;
**LR = Low Range, MR = Medium Range and HR = High Range; in the case of the two 5 Hach solutions reagent “A” is the molybdate reagent and reagent “B” is the amino acid reagent.

In addition to the measurements summarised in table 1, the water analysing device 100 may also be adapted for measuring hydrogen sulphide and sulphide concentrations, for example.

The water analysing device 100 is adapted for the performance of onsite photometric measurements, therefore it is appropriately waterproof, provided with thermal insulation and may be heated.

The advantage of the sealed (with septum or cap) cuvette 4 used in the water analysing device 100 is that the compositions of the heat-sensitive or volatile reagents do not change, they do not evaporate, and do not spill, and they are also protected from environmental impacts (e.g., humid environment), thereby contributing to the precision of the measurements. In addition to this, as a result of the multiple cuvettes 4 located in the water analysing device 100, a new cuvette 4 may be used for each photometric measurement, preventing with this the build-up of contamination on the permanent surfaces. The cuvettes 4 must be stored and because of the requirement for simpler servicing it must also be easy to replace them; a solution for this is the replaceable tray 2, which may be easily pulled out on the first rail 3 and a replaceable tray 2 filled with new, clean cuvettes 4 may be put in its place.

The advantages of the present invention include that, as opposed to the water analysing devices performing continuous measurements currently on the market, using the water analysing device 100 according to the present invention measurements may be made only at the required frequency, in addition it provides results faster than certain laboratory measurements, and is more cost-efficient at the same time, because unnecessary measurements are not performed and the measurement of the sample takes place onsite, i.e. at the place where the sample was taken.

Due to its analyser the water analysing device 100 according to the present invention is adapted for the photometric measurement of multiple parameters. Most currently known water analysing devices are adapted for the measurement of one parameter, so in order for a wastewater treatment plant to be able to examine the scale of parameters examined by the present invention using the devices known according to the state of the art it would be necessary to use several different devices. In other words, the water analysing device 100 according to the present invention is a device adapted for the photometric measurement of multiple parameters, in other words it can save significant costs for, e.g., wastewater treatment plants.

The data and results provided by the water analysing device 100 according to the present invention may also be integrated into other systems, thereby contributing to a more comprehensive picture being created about, for example, the operation of a wastewater treatment plant or about a given natural body of water.

There are no electrodes or flow-through cells in the water analysing device 100 according to the present invention, which require special attention from the point of view of contamination. It may be generally said of electrode measurements that they are imprecise and unreliable. Sediment, discolouration and blockages may occur in flow-through cells, which problems may have a negative impact on the reliability of the measurement results. A good solution for overcoming the above problems is the use of one cuvette for each measurement, which may be collected and regenerated after use. If a sealed cuvette is used for the measurement, the reagents are measured into it in advance, thereby reducing the reagent dispensing requirement. The device periodically launches an automatic cleaning protocol for the purpose of cleaning the tubing of the water analysing device 100. The purpose of the cleaning protocol is to sterilise the tubing of the water analysing device 100, to remove persistent microbes, biofilm algae, etc., this usually means rinsing with bleach. The cleaning fluid (bleach) tank is not depicted.

In other words, the significant advantage of the water analysing device 100 according to the present invention as compared to the water analysing devices according to the state of the art is that by using the device according to the present invention photometric measurement may be performed onsite, in other words the water analysing device according to the present invention is able to perform precise measurements even without laboratory conditions or human intervention. Due to the fact that the measurement of the samples is performed actually in the field, next to or at least close to the examined water, the data supply, the warnings, predictions and so the intervention process are all faster.

1. Water analysing device (100) for the photometric analysis of samples taken from natural or artificial bodies of water, characterised by that it contains the followings:
a frame, covered by thermal insulation, in which fans are used to ventilate the interior space;
tubes between the individual elements of the water analysing device for transporting the fluids;
sampler for taking samples from the natural or artificial bodies of water;
one or more sampling pumps for transporting fluids, for sucking up the water from the sampler;
one or more filters (1) connected to the sampler and the sampling pump for filtering out the shaped bodies to be found in the samples;
one or more buffer tanks connected to the sampling pump;
one or more needles or dispensing units for dispensing the filtered sample, the reagents and the solvents;
one or more sample dispensing pumps connected to the buffer tank;
one or more reagent dispensing pumps connected to the reagent tanks;
one or more sample and reagent dispensing positions (9) connected to the sample and reagent dispensing pumps;
one or more cuvettes (4);
robotic arm (6) for moving the one or more cuvettes (4), on which robotic arm (6) there is a gripping arm (7), and which gripping arm (7) is adapted to grasp the cuvettes (4);
replaceable tray (2) for storing the one or more cuvettes (4);
a stirring unit (10) for stirring the sample and the reagents;
a measuring cell (11) for the photometric analysis of the sample in the one or more cuvettes (4);
source of electricity for operating the components operating with electricity.

2. Water analysing device (100) according to claim 1, characterised by that there is more than one cuvette (4) in the water analysing device (100).

3. Water analysing device (100) according to claim 1, characterised by that the sampler preferably has an adjustable length and/or a silicone tube connecting it to the one or more filters (1) via a sampler pump.

4. Water analysing device (100) according to claim 1, characterised by that the filter insert of at least one of the one or more filters (1) is metal or characterised by that the covering of the water analysing device (100) is provided with thermal insulation.

5. (canceled)

6. Water analysing device (100) according to claim 1, characterised by that the number of cuvettes (4) is between 120 and 240.

7. Water analysing device (100) according to claim 1, characterised by that at least some of the cuvettes (4) in the water analysing device (100) are sealed with a septum or a cap.

8. Water analysing device (100) according to claim 1, characterised by that the gripping arm (7) of the water analysing device (100) is provided with a light source and a sensor located opposite each other in such a way that when the cuvette (4) is grasped the light emitted by the light source gets to the sensor via the cuvette (4).

9. Water analysing device (100) according to claim 1, characterised by that it contains a heated digester (12) or characterised by that it contains a cap screwing unit (8) adapted for removing and replacing the cap of the cuvette (4).

10. (canceled)

11. Water analysing device (100) according to claim 1, characterised by that it has a filter (1) and the filter insert of the filter (1) has holes with a diameter of 25-80 μm.

12. Water analysing device (100) according to claim 1, characterised by that it has two filters (1), where the filter insert of the first filter (1) according to the flow of liquid has holes with a diameter of 25-80 μm and the filter insert of the second filter (1) has holes with a diameter of 0.4-25 μm.

13. Water analysing device (100) according to claim 1, characterised by that the one or more filters in the water analysing device (100) are provided with a pressure sensor.

14. Water analysing device (100) according to claim 1, characterised by that the replaceable tray (2) of the water analysing device (100) contains three plates, where the lower plate is continuous in structure, and the two upper plates contain bores (13) for accommodating the cuvettes (4), and said bores (13) have a conical shape.

15. Water analysing device (100) according to claim 1, characterised by that the number of needles, or of the dispensing positions is between 1 and 10, preferably 6 or characterised by that the number of pumps is between 2 and 10, preferably 6.

16. (canceled)

17. Water analysing device (100) according to claim 1, characterised by that the number of wavelengths measured in the measuring cell (11) is between 1 and 8, preferably 4.

18. Measuring cell (11) for performing the photometric measurement of samples taken from natural or artificial bodies of water characterised by that it contains the followings:
a broad spectrum light source;
a light source aperture positioned directly in front of the light source;
an aspherical collimator lens positioned in front of the light source aperture;
an optical filter changer;
a semi-transparent mirror;
a measuring branch aperture located behind the semi-transparent mirror;
a cuvette located behind the measuring branch aperture;
a transmission detector of a size larger than a measuring beam located behind the cuvette;
a reference aperture positioned at 45° next to the semi-transparent mirror;
a reference detector of a size greater than a reference beam behind the reference aperture.

19. Method for performing the photometric analysis of samples taken from natural or artificial bodies of water using the water analysing device (100) according to claim 1, characterised by that the method contains the following steps:
a) before sampling, removing the stagnant water from the water analysing device (100), then rinsing the sampler with adjustable length and the one or more filters (1) with the sample to be measured;
b) sampling;
c) filtering the sample obtained in step b) through the one or more filters (1);
d) the rinsing of the buffer tank supplied with a level indicator, the sample dispenser (11) and the section between the two with the filtered sample to be measured obtained in step c);
e) grasping the cuvette (4) with the gripping arm (7) and checking if the cuvette (4) is open or sealed;
f) the transporting of the cuvette (4) gripped by the grasping arm (7) with the robotic arm (6) to the given position, if necessary, the screwing off of the cap of the cuvette (4) sealed with a cap and the transporting of the cuvette (4) to the sample dispensing position or pushing the cuvette (4) sealed with a septum onto the sample dispensing needle;
g) optionally the dispensing of demineralised water or other reagent into the cuvette (4) at the sample or reagent dispensing position;
h) screwing the cap onto open cuvettes (4) without caps;
i) homogenising the sample in the cuvette (4);
j) optionally digesting the sample in the cuvette (4) at a temperature higher than the ambient temperature using the heated digester (12);
k) after a predetermined waiting time has elapsed the cuvette (4) is placed in the measuring cell (11) by the gripping arm (7) and the robotic arm (6), where the photometric measurement of the sample in the cuvette (4) takes place.

20. Method for performing the photometric analysis of samples taken from natural or artificial bodies of water according to claim 19, characterised by that the temperature applied in step i) is between 120° C. and 180° C., preferably 150° C., and/or the digesting duration is 30-140 minutes, preferably 30 minutes.

21. Method for performing the photometric analysis of samples taken from natural or artificial bodies of water according to claim 19, characterised by that a clean cuvette (4) is used for each sample.

22. Method for performing the photometric analysis of samples taken from natural or artificial bodies of water according to claim 19, characterised by that the parameters selected from the following list are determined photometrically: phosphate ions; nitrite ions; nitrate ions; ammonium ions; chemical oxygen demand, dissolved hydrogen sulphide, sulphides.

23. Water analysing system that includes the water analysing device (100) according to claim 1, a unit capable of GSM communication and a webserver.
